(19) United States
(12) Patent Application Publication
(10) Pub. No.: US 2006/0149140 A1
(43) Pub. Date: Jul. 6, 2006

(54) Automated system for patient diagnosis and crisis management system

(76) Inventor: Paulla Eldridge, Peoria, AZ (US)

Correspondence Address: KUNZLER & ASSOCIATES, 8 EAST BROADWAY, SUITE 600, SALT LAKE CITY, UT, 84111, US

(21) Appl. No.: 11/030,499

(22) Filed: Jan. 6, 2005

(51) Int. Cl. A61B 5/00

(52) U.S. Cl. 600/300; 128/920; 128/923

(57) ABSTRACT

An apparatus, system, and method are disclosed for determining a patient's condition. A system for determining a patient's condition, in one embodiment, comprises a computer processor device, an interface between the computer processor and one or more patient monitoring devices, and a software program configured to interpret patient input data, diagnose the patient's condition, and recommend treatment options. A method for determining a patient's condition may comprise receiving patient data from one or more patient monitoring devices, consulting a treatment database, determining a diagnosis, and converting a diagnosis into a treatment.

### BACKGROUND OF THE INVENTION

1. Field of the Invention

This invention relates to medical systems and more particularly relates to apparatus, systems, and methods for diagnosing a patient's condition.

2. Description of the Related Art

Healthcare providers have a common goal to provide an effective medical treatment for patient illnesses. Often multiple medical attendants are needed to administer an effective treatment, particularly over an extended period of time. However, in order to provide affordable healthcare, and due to time restrictions placed on professionals, treatments are often administered by medical staff with limited training and knowledge of medical conditions. Occasionally, even trained practitioners, such as doctors, may lack the necessary background and training to effectively diagnose and treat certain medical conditions.

Specifically, in cases regarding acute medical conditions, doctors may not have the time or the resources to research a medical condition in depth before recommending a diagnosis or treatment. As a result, patients may unduly suffer from a treatable condition. In extreme cases, a mistreated medical condition may worsen and/or cause death.

Proper medical treatment administered in a timely manner may decrease the amount of time a patient suffers and may save an individual's life. Generally, a patient's condition changes over the course of a treatment. Unfortunately, a skilled professional, such as a doctor or a specialist, may not be readily available to continually assess a patient's needs. Consequently, less-skilled professionals typically perform regular treatments. Medical attendants, however, may not be trained to recognize changes in certain medical conditions or to recognize when an unsuitable diagnosis or treatment is initially rendered. In many cases, the medical attendant may not be authorized to alter or prescribe a treatment even if the medical attendant is aware of a specific patient need. Consequently, a patient may be required to wait an indefinite amount of time for a doctor or a specialist to become available. Furthermore, the patients may be unnecessarily subjected to improper treatments that harm the patient rather than cure the illness.

Medical equipment, monitoring devices, patient records, diagnostic software, informational databases, and the like may be useful in determining a patient's present condition and providing a proper treatment. However, helpful resources may not be readily available to medical attendants. For example, patient information may be stored in multiple databases that may not be available to medical attendants. As a result, treatments may be given without consideration to a patient's complete history. Moreover, the records may not be completely up-to-date because of limited access to the records.

From the foregoing discussion, it should be apparent that a need exists for an apparatus, system, and method that integrates current technologies such as monitoring devices and diagnostic software programs to determine a patient's condition and to recommend a suitable treatment. Beneficially, such an apparatus, system, and method would enable medical attendants to effectively treat a patient's medical condition by accessing a treatment database and determining a recommended treatment based on the patient's personal data. A doctor or specialist may further be consulted directly or through remote communication devices to provide guidance and authorization to perform a recommended treatment.

### SUMMARY OF THE INVENTION

The present invention has been developed in response to the present state of the art, and in particular, in response to the problems and needs in the art that have not yet been fully solved by currently available medical systems. Accordingly, the present invention has been developed to provide an apparatus, system, and method for determining a patient's condition that overcome many or all of the above-discussed shortcomings in the art.

A system for determining a patient's condition, in one embodiment, comprises a computer processor device, an interface between the computer processor and one or more patient monitoring devices, and a diagnostic software program. In one embodiment, the software program interprets patient input data, diagnoses the patient's condition, and recommends treatment options. To provide access to greater resources, the computer processor device may be adapted to connect to an internetwork and/or a hospital database. In certain embodiments, the computer processor device communicates with multiple databases including a treatment database.

The system may further include one or more input devices, which may include a keyboard, a microphone, a video camera, a touch-sensitive display device, a control panel, and the like. In addition, the system may include one or more output devices to communicate information to a user. The output devices may include a speaker, a display device, a printer, and the like.

The system may further include, in one embodiment, a speech recognition module to enable a user to communicate vocal commands to the computer processor device. In a further embodiment, a speech synthesis module may translate a computer command to a recognizable sound message for a user to hear. Additionally, a communication module may further enable wired or wireless communication with a remotely located entity, such as a doctor or a specialist.

In certain embodiments, the system further comprises a mobile conveyance device to mount and transport selected components of the system including the computer processing device. The mobile conveyance device may have compartments to store and transport patient monitoring devices and treatment devices. In one embodiment, the system includes a portable power source.

An apparatus for determining a patient's condition may include a computer processor device, a control panel, and a software program. The control panel may provide an interface between the computer processor and one or more patient monitoring devices. Consequently, the user may be able to direct an integrated system for determining a patient's condition.

The control panel of the apparatus may comprise one or more displays to indicate a current status of the computer processor device. One or more switches, which may comprise at least one key switch, may provide the user manual access to the computer processor device. The control panel may further include a security mechanism to provide selective access to the computer processor device.

In one embodiment, a computer readable storage medium is provided for conducting a method of interpretation of medical data. The method may comprise receiving patient medical data from a monitoring device and interpreting patient medical data, consulting a treatment database, and automatically converting interpreted patient medical data into a diagnosis. The computer readable storage may further comprise conducting a determination of appropriate medical treatments and communicating of treatment information.

A method for determining a patient's condition may comprise receiving patient data from one or more patient monitoring devices, consulting a treatment database via a software program, determining a diagnosis, and converting a diagnosis into a treatment. The method may further comprise communicating a treatment to medical attendants.

A physician's treatment recommendation may be received, either in person or through communication devices, and may determine the treatment. In addition, treatment activities may be recorded and monitored to determine the efficacy of the treatment. The treatment recommendations may be altered based on monitored results.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

The present invention enables medical attendants to determine a patient's condition with the help of diagnostic software and monitoring devices. A remotely located physician may contribute to a patient's diagnosis and treatment though communication devices. Furthermore, patient records may be integrated and updated in conjunction with a diagnosis and treatment. These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a perspective view illustrating one embodiment of a diagnosis and treatment unit of the present invention;

FIG. 2 is a front view of one embodiment of a control panel in accordance with the present invention;

FIG. 3 is a functional block diagram illustrating one embodiment of a patient diagnosis and treatment system of the present invention; and

FIG. 4 is a flow chart diagram illustrating one embodiment of a patient diagnosis and treatment method of the present invention.

### DETAILED DESCRIPTION OF THE INVENTION

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

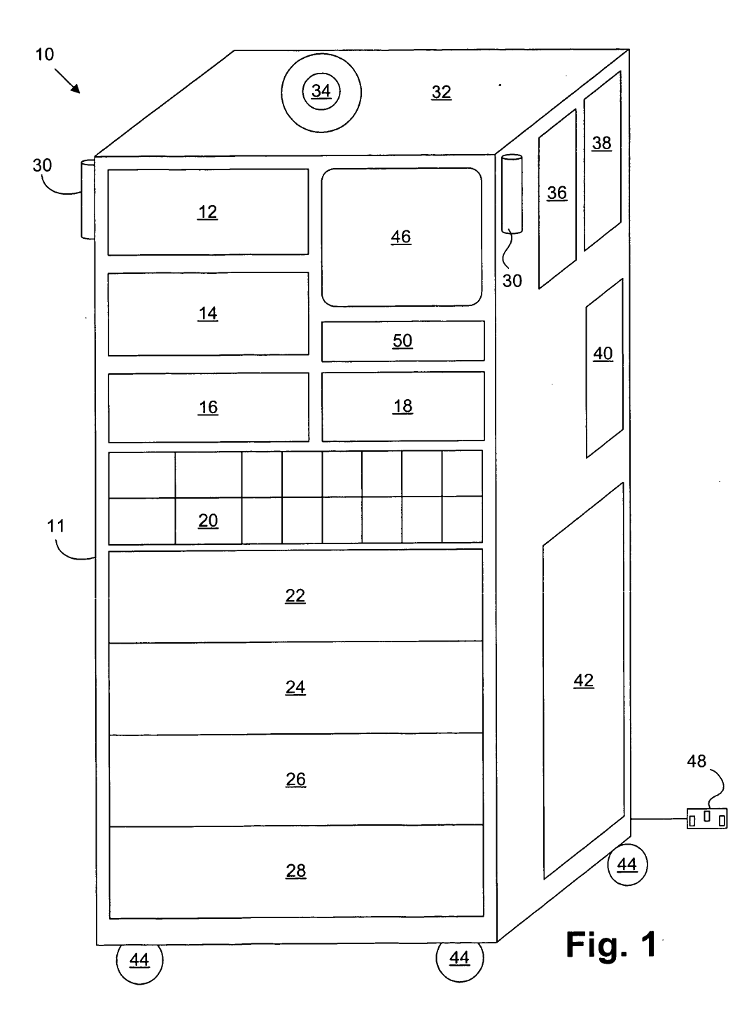

FIG. 1 illustrates one embodiment of a diagnosis and treatment unit 10 of the present invention. The diagnosis and treatment unit 10 may include a computer processing device having software modules that interpret a patient's condition based on inputs from medical devices, monitors, medical records, and/or medical attendants. The diagnosis and treatment unit 10, in the depicted embodiment, is completely self-contained and is adapted to connect to a hospital's computer network or to the Internet using wired or wireless interfaces.

The diagnosis and treatment unit 10 may be mounted in a cart 11 with a corded connection 48 for 110 Volt AC power and optional DC backup sources. All other devices may be powered from the cart 11. In certain embodiments, the diagnosis and treatment unit 10 may be battery-powered and cordless. In one embodiment, the cart 111 is provided with wheels 44. The depicted wheels 44 are 4″ locking, swiveling, non-skid, non-conducting wheels 44. In an alternative embodiment, the diagnosis and treatment unit 10 may be carried in a backpack or mounted to a powered vehicle. A variety of devices that range in size and adaptability may be used to support and transport the diagnosis and treatment unit 10. A smaller diagnosis and treatment unit 10 may provide greater mobility, while a larger diagnosis and treatment unit 10 may provide greater utility.

A control panel 50 may provide an interface between the computer processor device and one or more patient monitoring devices. In addition, the diagnosis and treatment unit 10 may include one or more input devices, such as a keyboard 18 and a touch-screen LCD computer monitor 46. The monitor 46 may be used for displaying patient diagnosis, treatment instructions, physician communications, database queries, or the like. The computer keyboard 18 may facilitate user input. Of course, other input devises, such as a camera 34, may be adapted for multiple purposes.

In one embodiment, the diagnosis and treatment unit 10 includes a cardiac monitor 12 with a paddle-less cardiac pacemaker/defibrillator 14. Alternatively, the cardiac monitor 12 and paddle-less cardiac pacemaker/defibrillator 14 may be combined in one unit, such as the PD 1400 manufactured by Zoll Medical Corporation of Burlington, Mass. In alternative embodiments, the diagnosis and treatment unit 10 may include a variety of monitoring devices useful to diagnose and treat a patient.

The depicted cart 11 may provide a plurality of slots and/or drawers sized to fit various components associated with diagnosing and treating a patient. In one embodiment, a utility drawer 16 holds devices such as a cordless headset. Headsets may enable a user to communicate with the diagnosis and treatment unit 10, or optionally to participate in remote consultations with physicians via telephone or the Internet.

The slots illustrated in the depicted embodiment may further include computerized drug dispensing drawers 20, such as those manufactured by Pyxis Corporation of San Diego, Calif. In one embodiment, the drug dispensing drawers 20 are programmed to open automatically upon completion of a diagnosis. One or more computerized intravenous drug dispensing drawers 22 may additionally be programmed to open automatically upon diagnosis. The drawers 24, 26, and 28 may be miscellaneous hospital supply drawers.

The diagnosis and treatment unit 10 may be suitable to mount a variety of medical equipment. In one embodiment, brackets 30 may be used to mount intravenous poles. The slots 36, 38, and 40 may contain equipment such as the i-STAT 1 non-invasive blood chemistry analyzer manufactured by i-STAT Corporation of East Windsor, New Jersey, and the vital signs monitor and blood pressure cuff system manufactured by Critikon/GE Medical Systems of Tampa, Fla. Other equipment that may be located in the slots 36, 38, and 40 may include output devices such as printers for creating a hard-copy record of patient monitoring results and/or attendant actions. A larger slot 42, in one embodiment, may be located on both sides of the diagnosis and treatment unit 10 and may be reserved for attaching medical equipment, such as radiology equipment.

The surface 32 of the diagnosis and treatment unit 10 may consist of a non-skid and non-conductive material for protective purposes. A built-in digital camera 34 may be mounted on the surface 32 and may be used for Internet-based communication with a physician. The diagnosis and treatment unit 10 enables medical attendants to have multiple resources at hand to help diagnose and treat a patient.

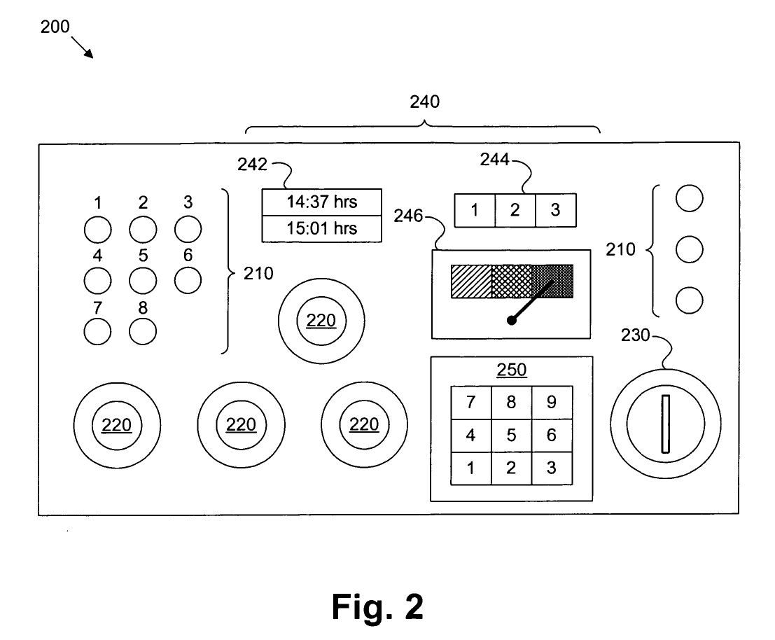

FIG. 2 illustrates in greater detail one embodiment of a control panel 200. As depicted, the control panel 200 includes light indicators 210, switches 220, a key switch 230, displays 240, and a security mechanism 250. The control panel 200 may be configured to reflect selected monitoring devices and systems corresponding to a particular embodiment of the diagnosis and treatment unit 10. Thus, any combination of controls, displays, security mechanisms, and/or switches that may be used with a computer processor device is within the scope of the present invention.

The light indicators 210 may indicate certain functions such as a power on, battery strength, system ready, auxiliary systems ready, drug tray loaded, auto system on, auto system hold, facility network on line, internet connection established, remote access established, remote system hold, system malfunction, and the like. The light indicators 210 may provide fail-safe and system status indicators to inform a user of the status of the diagnosis and treatment unit 10 and related systems.

Switches 220 may provide a variety of safety features and may enable a user to manually override the systems. Examples of switches 220 may include but are not limited to a system reset, emergency shut down, remote initiation of system override, manual push pull hardwired local emergency stop, local hold override, remote hold override, local diagnosis confirm/override, system input lock out, system start cycle, and the like.

A key switch 230 configured to provide selective access may additionally enable a manual override in certain embodiments. In one embodiment, a key switch 230 provides an initial level of security to prevent unauthorized access to the systems. Alternatively, a key switch 230 may permit access to selected systems within the overall system. A key switch 230 may further be used in conjunction with one or more other security mechanisms 250 to verify user identification.

Displays 240 may inform the user of the status of current procedures. For example, a clock 242 may indicate a start and stop time for a diagnosis or treatment. A cycle use counter 244 may indicate a current cycle. A system input lockout 246 may indicate the level of activity of a certain system.

In addition, one or more security mechanisms 250 may prevent unauthorized access to the systems. In certain embodiments, the security mechanism 250 may comprise a code pad, a card scanner, a voice recognition module, or the like. Other mechanisms associated with monitoring devices and communications systems may be included to further enhance the security and safety of the diagnosis and treatment unit 10.

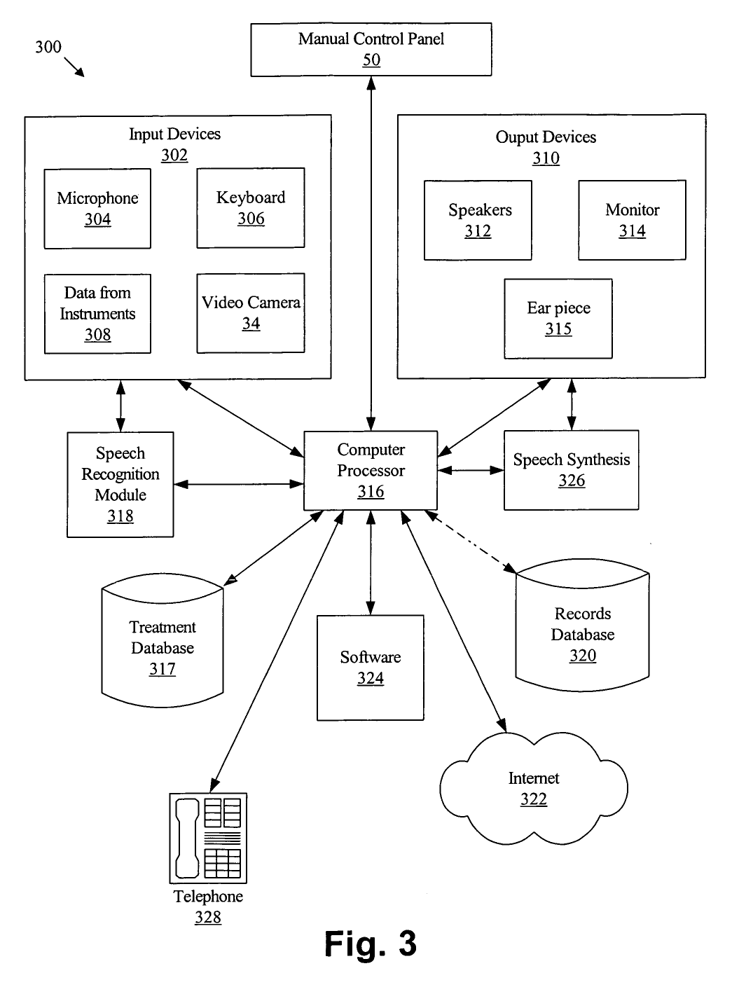

FIG. 3 is a schematic block diagram depicting various functional components of one embodiment of a patient diagnosis and treatment system 300. As illustrated, the diagnosis and treatment system 300 includes input devices 302, output devices 310, a computer processor 316, a control panel 50, a treatment database 317, a speech recognition module 318, a records database 320, a network such as the Internet 322, diagnostic software 324, a speech synthesis module 326, and a telephone 328.

The computer processor 316 may be any suitable logic device, including a microprocessor, a microcontroller, an application specific digital processor, or even discrete digital logic components. In one embodiment, the computer 316 operates upon diagnostic software 324 which may be resident in RAM or ROM memory. As depicted, the computer processor 316 is configured to communicate with and, in some cases, control the various other modules of the diagnosis and treatment system 300.

The input devices 302 may include a microphone 304. In one embodiment, the microphone 304 is integrated into a wireless headset for user convenience. The speech recognition module 318 may interpret spoken commands for the diagnostic software 324. A keyboard 306 may be used to provide direct input, including patient symptoms entered by medical personnel. The instruments or other medical devices 308 may include heart monitors, blood pressure monitors, and the like that are connected to the patient in order to monitor conditions such as heart rate, blood pressure, temperature, etc. The input devices 302 may also include a digital video camera 34, which enables personnel such as a doctor communicating remotely to view the patient, patient information, and a treatment being given.

In one embodiment, the patient diagnostic software 324 interprets all input data including those directly input by attendant medical personnel and those received from medical devices 308. The diagnostic software 324 then consults the treatment database 317, and employs an algorithm to select the appropriate treatment that matches the symptoms received from the instruments and medical devices 308 and the user inputs. Databases may include authoritative standards for medical code situations, such as those promulgated by the American Heart Association. In one embodiment, the treatment database 317 is updateable at regular intervals over the Internet 322. The treatment database 317 and/or the diagnostic software 324 may be configured to handle many or all types of critical situations and traumas. Consequently, the diagnosis and treatment system 300 may be adapted to the trauma room.

The diagnosis and treatment system 300, in one embodiment, is connected to the patient record database 320 through an Internet connection 322. The diagnosis and treatment system 300 may use this information to form diagnosis and treatment recommendations. Users may also access patient medication records and other useful information from the patient record database 320. In one embodiment, the treatment database 317 contains a complete drug handbook and a drug calculator. The diagnostic software 324 may in one embodiment be configured to warn of a drug interaction before a recommended drug is administered.

In one embodiment, the diagnosis and treatment system 300 is also configured to provide users with diagnosis and treatment recommendations through the output devices 310, which, in the depicted embodiment, include a monitor 314, speakers 312, and wireless earpieces 315. A speech synthesis module 326 may be employed to create audio prompts that are provided to the users with the output devices 310. The monitor 314, in one embodiment, may provide a plurality of visual segments, such as individual windows. Each segment may remain resident on the monitor 314 during use, or may be configured to appear temporarily and close after a specified time. For instance, one segment may display a patient chart, including lab and test results, as well as other patient records. Another segment may display pharmacology data, such as information on the patient's drugs and any contraindications that could occur with a recommended treatment. Other segments may further display on-line databases, remote communications, medical code information, diagnostic information, treatment recommendations, and the like. In one embodiment, the monitor 314 is touch-sensitive to receive input from a user.

In one embodiment, the diagnosis and treatment system 300 is also configured to display information regarding drugs that may be prescribed, such as pictures of the drug packaging and proper dosages. The monitor 314 may be configured to display hyperlinks to explanations of a procedure to be performed and/or a drug to be administered. As discussed above, the diagnosis and treatment unit 10 may be configured to automatically open the particular drawer containing a prescribed drug once a drug has been recommended by the diagnosis and treatment system 300.

In one embodiment, the diagnosis and treatment system 300 supports communication with one or more remotely located physicians. In one embodiment, direct communication is achieved by utilizing the digital video camera 34, the monitor 314, the speakers 312, and the microphones 304. Alternatively, other communication methods may be employed. Beneficially, the physician may remotely review medical data, view the patient, access medical records, communicate instructions to attendants, and the like.

In one embodiment, one attendant may be designated as the “captain.” The captain may be, for instance, an emergency room doctor or a charge nurse. The captain may have controlled access to all communication channels and the ability to issue commands to the diagnosis and treatment system 300. Command status light indicators 210 may indicate who currently controls of the diagnosis and treatment system 300. In this embodiment, other attendants may be restricted from issuing commands to the diagnosis and treatment system 300 and may not be able to receive selected communication channels or information from select databases.

The schematic flow chart diagram that follows is generally set forth as a logical flow chart diagram. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Additionally, the order in which a particular method occurs may or may not strictly adhere to the order of the corresponding steps shown.

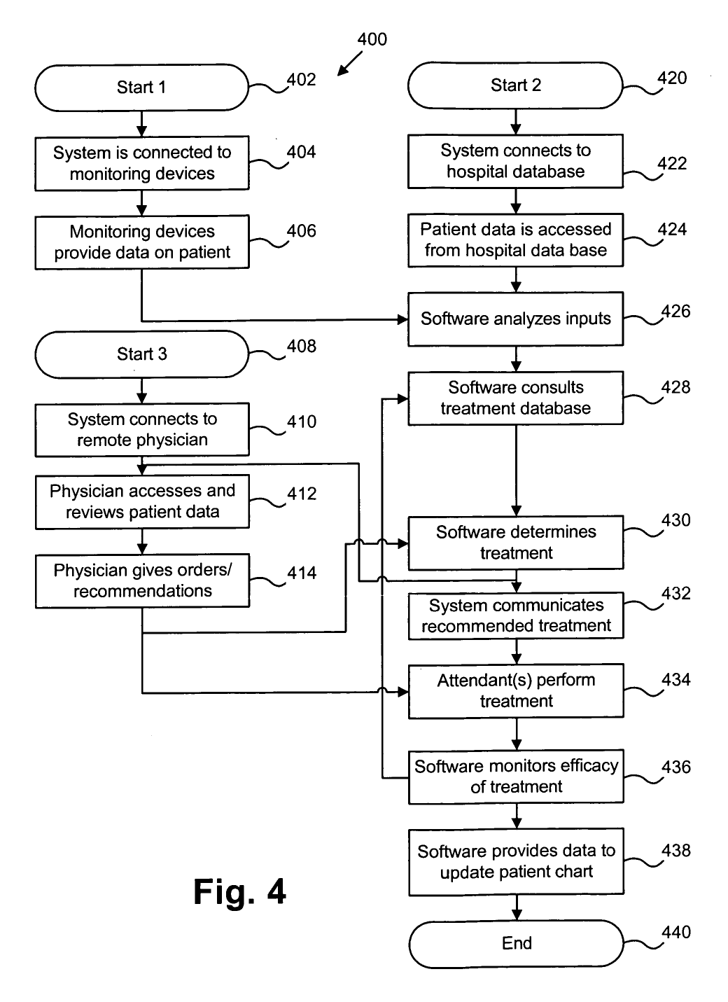

FIG. 4 depicts one embodiment of a diagnosis and treatment method 400 for implementing the diagnostic software 324. The diagnosis and treatment method 400 has a main procedure 402 and two optional procedures 408, 420. The diagnosis and treatment method 400 starts 402 and the diagnosis and treatment unit 10 is connected 404 to a plurality of patient monitoring devices. The diagnosis and treatment unit 10 then receives patient data 406 from the patient monitoring devices.

At this point, optional steps 422, 424 of the diagnosis and treatment method 400 may start 420. The diagnosis and treatment system 300 is then connected 422 to the hospital's network. The diagnosis and treatment system 300 subsequently accesses 424 patient information, such as charts, lab reports, x-rays, and other desired records. Returning to the main diagnosis and treatment method 400, the diagnostic software 324 then analyzes 426 data inputs, such as monitoring device inputs, attendant personnel direct input, and patient record inputs. The software 324 then consults 428 the treatment database 317. The diagnostic software 324 then uses a sophisticated algorithm and the treatment database 317 to determine the recommended treatment for the patient 430.

In one embodiment, the diagnostic software's 324 diagnosis and treatment recommendation is communicated 432 to attendants via visual or audio prompts. The attendants may perform the recommended treatment 434 while receiving instructions from the diagnosis and treatment system 300 or the remote physician. Attendants may use medical devices or medications included on the diagnosis and treatment unit 10. Attendants may also use input devices 302 to command the diagnosis and treatment system 300 to provide additional treatment details. In one embodiment, the diagnosis and treatment system 300 is configured to provide the attendants with useful diagnostic indications and proper treatment procedures by prompting the attendant to test for diagnostic signs or giving step-by-step instructions for performing a procedure.

During and after treatment, the diagnostic software 324 monitors 436 the efficacy of the treatment. The diagnostic software 324 may subsequently determine additional treatment is required and again consult 428 the treatment database 317 and determine additional or alternative treatments 430.

In one embodiment, alternative steps 410,412,414 are used to allow a remote physician to communicate with the diagnosis and treatment system 300 and attendant personnel. This embodiment of the diagnosis and treatment method 400 starts 408 and connects 410 to physicians over the Internet 322 or by telephone 328. The physician may access 412 patient data, the system 300 diagnosis and treatment recommendation, or other information. The physician may then override the software's diagnosis or provide additional instructions to attendants 414. The physician's remote participation may come at any time during the diagnosis and treatment method 400, but is shown communicating after steps 430 and 432. When the software 324 determines 436 that the treatment has been successful or is completed, the diagnostic software 324 may in one embodiment provide information for updating 438 the patient's chart either locally or directly to the hospital's records database 320. The method 400 may end 440 when treatment is complete and patient records have been updated.

In one embodiment, the diagnostic software 324 records all actions taken by attendant personnel and the diagnosis and treatment system 300. The diagnostic software 324 may log data inputs, attendant personnel conversations, and diagnostic software 324 recommendations. The digital video camera 34 may also record the treatment performed. The complete treatment record may be uploaded to the hospital's records database 320.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for determining a patient's condition, the system comprising:
a computer processor device;
an interface between the computer processor and one or more patient monitoring devices; and
a software program configured to interpret patient input data, diagnose the patient's condition, and recommend treatment options.

2. The system of claim 1, wherein the computer processor device is adapted to connect to an internetwork.

3. The system of claim 1, wherein the computer processor device is configured to communicate with a hospital database.

4. The system of claim 1, wherein the computer processor device is configured to communicate with a treatment database.

5. The system of claim 1, further comprising at least one input device configured to receive input from a user.

6. The system of claim 5, wherein the at least one input device is selected from the group consisting of a keyboard, a microphone, a video camera, a touch-sensitive display device, and a control panel.

7. The system of claim 1, further comprising at least one output device configured to communicate information to a user.

8. The system of claim 7, wherein the at least one output device is selected from the group consisting of a speaker, a display device, and a printer.

9. The system of claim 1, further comprising a speech recognition module configured to communicate vocal commands to the computer processor device.

10. The system of claim 1, further comprising a speech synthesis module configured to translate a computer command to a recognizable sound message.

11. The system of claim 1, further comprising a communication module configured to enable wired or wireless communication with a remotely located entity.

12. The system of claim 1, further comprising a mobile conveyance device configured to mount and transport selected components of the system including the computer processing device.

13. The system of claim 12, wherein the mobile conveyance device comprises compartments to store and transport patient monitoring devices and treatment devices.

14. The system of claim 12, wherein the mobile conveyance device is selected from the group consisting of a backpack, a cart, and a powered vehicle.

15. The system of claim 1, further comprising a portable power source.

16. An apparatus for determining a patient's condition, the apparatus comprising:
a computer processor device;
a control panel configured to provide an interface between the computer processor and one or more patient monitoring devices; and
a software program configured to interpret patient input data, diagnose the patient's condition, and recommend treatment options.

17. The apparatus of claim 16, wherein the control panel comprises one or more displays to indicate a current status of the computer processor device.

18. The apparatus of claim 16, wherein the control panel further comprises one or more switches configured to provide manual access to the computer processor device.

19. The apparatus of claim 18, wherein the one or more switches comprises at least one key switch.

20. The apparatus of claim 16, wherein the control panel further comprises a security mechanism configured to provide selective access to the computer processor device.

21. The apparatus of claim 16, further comprising a mobile conveyance device configured to mount and transport the computer processing device and the control panel.

22. The apparatus of claim 21, wherein the mobile conveyance device comprises compartments to store and transport patient monitoring devices and treatment devices.

23. The apparatus of claim 21, wherein the mobile conveyance device is selected from the group consisting of a backpack, a cart, and a powered vehicle.

24. A computer readable storage medium configured to contain computer code for conducting a method of interpretation of medical data, the method comprising:
receiving patient medical data from a monitoring device; and
interpreting patient medical data;
consulting a treatment database; and
automatically converting interpreted patient medical data into a diagnosis.

25. The computer readable storage medium of claim 16, wherein the method further comprises conducting a determination of appropriate medical treatments.

26. The computer readable storage medium of claim 16, wherein the method further comprises configured communicating of treatment information.

27. A method for determining a patient's condition, the method comprising:
receiving patient data from one or more patient monitoring devices;
consulting a treatment database via a software program configured to interpret patient input data, diagnose the patient's condition, and recommend treatment options;
determining a diagnosis; and
converting a diagnosis into a treatment.

28. The method of claim 15, further comprising communicating a treatment to medical attendants.

29. The method of claim 15, further comprising receiving a physician's treatment recommendations.

30. The method of claim 15, further comprising recording treatment activities and monitoring the efficacy of the treatment.

31. The method of claim 22, further comprising altering the treatment recommendations based on monitored results.
